(19) United States
(12) Patent Application Publication
(10) Pub. No.: US 2024/0262780 A1
(43) Pub. Date: Aug. 8, 2024

(54) PREPARATION OF AN INTERMEDIATE IN THE SYNTHESIS OF 2-CYCLODODECYL-1-PROPANOL

(71) Applicant: MILTITZ AROMATICS GMBH, BITTERFELD-WOLFEN (DE)

(72) Inventors: STEFAN MUELLER, LEIPZIG (DE); ANDREAS PETRI, LEIPZIG (DE); JHONNY JIMENEZ-PINTO, HALLE (SAALE) (DE)

(73) Assignee: MILTITZ AROMATICS GMBH, BITTERFELD-WOLFEN (DE)

(21) Appl. No.: 18/565,528

(22) PCT Filed: May 24, 2022
(86) PCT No.: PCT/EP2022/063988

§ 371 Date: Nov. 30, 2023

(30) Foreign Application Priority Data
Jun. 3, 2021 (EP) 21177644.8

(51) Int. Cl. C07C 67/347; C07C 29/56; C07C 67/62

(52) U.S. Cl. CPC C07C 67/347; C07C 29/56; C07C 67/62; C07C 2601/20

(57) ABSTRACT

A method for preparing a tertiary alcohol having a formula (I)

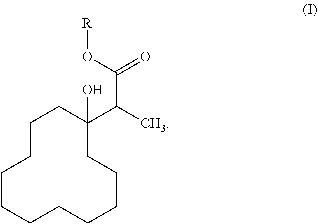

The method includes reacting cyclododecanone with an acrylate having a formula RO—CO—CH═CH₂ in a reductive aldol reaction under a metal catalysis. R in the formula (I) is a straight-chain C₁-C₈-alkyl, a branched C₁-C₈-alkyl, a straight-chain alkenyl group, a branched alkenyl group, an unsubstituted phenyl group, or a substituted phenyl group.

### CROSS REFERENCE TO PRIOR APPLICATIONS

This application is a U.S. National Phase application under 35 U.S.C. § 371 of International Application No. PCT/EP2022/063988, filed on May 24, 2022 and which claims benefit to European Patent Application No. 21177644.8, filed on Jun. 3, 2021. The International Application was published in German on Dec. 8, 2022 as WO 2022/253626 A1 under PCT Article 21(2).

### FIELD

The present invention relates to the preparation of a tertiary alcohol having the formula (I)

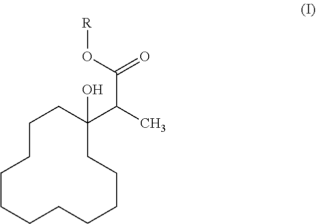

and to the preparation of 2-cyclododecyl-1-propanol proceeding from the intermediate prepared thereby.

### BACKGROUND

The commercially important aromatic substance 2-cyclododecyl-1-propanol (also known under the name Hydroxyambran) is conventionally prepared from cyclododecanone via two to three reaction steps, wherein, in the first step, cyclododecanone is reacted with an alkyl 2-bromopropionate, for example, ethyl 2-bromopropionate, in the presence of zinc (Reformatsky reaction) to form a tertiary alcohol. Elimination of water then leads to the corresponding unsaturated ester (a mixture of isomers), which is subsequently reduced to form 2-cyclododecyl-1-propanol.

This method is described in DE 37 03 585 A1.

Although the standard method provides the end product 2-cyclododecyl-1-propanol in good yields, the first reaction step, i.e., the Reformatsky reaction, is not only associated with relatively high material costs, but also involves extensive recovery or disposal operations and costs in that zinc bromide accumulates during the reaction.

An aspect of the present invention is to provide an improved method for preparing the intermediate in the synthesis of 2-cyclododecyl-1-propanol, namely, the tertiary alcohol from the Reformatsky reaction, which overcomes the aforementioned disadvantages.

### SUMMARY

In an embodiment, the present invention provides a method for preparing a tertiary alcohol having a formula (I)

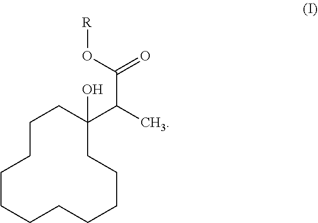

The method includes reacting cyclododecanone with an acrylate having a formula RO—CO—CH═CH₂ in a reductive aldol reaction under a metal catalysis. R in the formula (I) is a straight-chain C₁-C₆-alkyl, a branched C₁-C₈-alkyl, a straight-chain alkenyl group, a branched alkenyl group, an unsubstituted phenyl group, or a substituted phenyl group.

### DETAILED DESCRIPTION

The reaction can, for example, be performed under copper catalysis in the presence of a silane.

In an embodiment, polymethylhydrosiloxane (PMHS) can, for example, be used as the silane. Other silanes such as tetramethylhydrosiloxane, triethoxysilane or phenylsilane can also be used.

Cu(OAc)₂×H₂O can, for example, be used as the catalyst.

In an embodiment of the present invention, a phosphine compound is used as a ligand of the catalyst.

Triphenylphosphine can, for example, be used as the ligand.

In an embodiment of the present invention, a stabilizer is used for the acrylate.

Hydroquinone monomethyl ether can, for example, be used as the stabilizer for the acrylate.

In an embodiment of the present invention, the reaction can, for example, be performed in a solvent system.

The solvent system can, for example, include toluene or xylene.

The reaction can, for example, be performed at a temperature in the range from −30° ° C. to 80° C., for example, at a temperature in the range from 10° C. to 50° C., for example, in the range from 20° C. to 30° C.

In an embodiment of the present invention, R denotes a straight-chain or branched C₁-C₈-alkyl or -alkenyl group, for example, a methyl, ethyl, n-propyl, isopropyl, n-butyl, sec-butyl, tert-butyl, pentyl, hexyl, heptyl, octyl, 2-ethylhexyl, allyl or vinyl group. R can, for example, denote an ethyl, n-butyl or 2-ethylhexyl group.

The present invention further relates to the preparation of 2-cyclododecyl-1-propanol, comprising preparing of a tertiary alcohol having the formula (I) as set forth above, and converting the tertiary alcohol having the formula (I) so prepared to 2-cyclododecyl-1-propanol.

Instead of the Reformatsky reaction with zinc, which is not optimal in terms of both material costs and wastewater problems, a reductive aldol reaction under metal catalysis, particularly copper catalysis, is used in accordance with the present invention for the first step in the synthesis of 2-cyclododecyl-1-propanol.

The method according to the present invention can be represented schematically as follows:

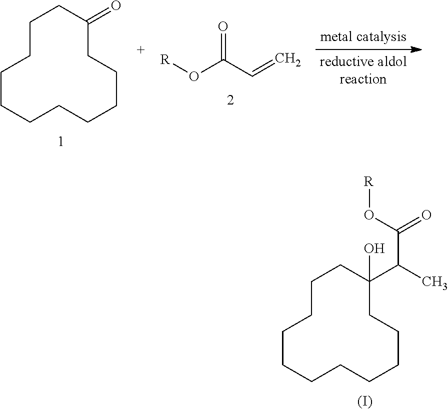

As can be seen from the above reaction scheme, the reductive aldol reaction of cyclododecanone 1 with the acrylate 2 leads to the tertiary alcohol (I).

The tertiary alcohol of formula (I) that is obtained corresponds to the product from the Reformatsky reaction as described in DE 37 03 585 A1 and can then be used to prepare 2-cyclododecyl-1-propanol.

The method according to the present invention provides the tertiary alcohol having the formula (I) in a stable form and in a yield of more than 75%.

Various metal catalysts are suitable for catalyzing the reductive aldol reaction, for example, catalysts based on rhodium, palladium, cobalt or zinc, as described in the literature in connection with this type of reaction. Copper catalysts, and in particular the compound Cu(OAc)₂×H₂O, have, however, proved particularly advantageous in the context of the present invention.

In the case of copper catalysis, the reductive aldol reaction generally takes place in the presence of a silane. In the case of other metal catalysts, such as rhodium compounds, for example, the reaction can also be performed in a hydrogen atmosphere as a reducing agent.

If the reductive aldol reaction is performed under copper catalysis in the presence of a silane, the O—Si bond of the siloxane compound that is formed in an intermediate step is usually cleaved with acid in order to arrive at the tertiary alcohol having the formula (I). Other reagents are, however, also possible for this cleavage reaction, such as fluorine compounds, e.g., ammonium fluoride or tetrabutylammonium fluoride (TBAF). In an embodiment of the present invention, HCl, H₂SO₄ or H₃PO₄ can, for example, be used as the acid. Other acids such as HNO₃ or para-toluenesulfonic acid can also be used. Bases such as sodium hydroxide solution and potassium hydroxide solution are also possible if the basic conditions remain mild, since the tertiary alcohol would otherwise break down.

In an embodiment of the present invention, ligands can, for example, be used for the catalyst, such as bidentate ligands as described in the literature in connection with reductive aldol reactions (Organic Letters, 2005, vol. 7, no. 19, pages 4225-4228; Beilstein Journal of Organic Chemistry, 2015, 11, pages 213 to 218; Tetrahedron Letters 47, 2006, pages 1403-1407). The ligand can also be selected from the group of organophosphorus compounds, such as phosphines, diphosphines, diamines or organic phosphites. The use of triphenylphosphine as the ligand has proved particularly advantageous in accordance with the present invention.

If a solvent system is used, the solvent system can, for example, include toluene or xylene and in one particular embodiment it consists entirely thereof.

In an embodiment of the method according to the present invention, a stabilizer, for example, hydroquinone monomethyl ether, can be used to reduce the undesired polymerization of acrylate. The undesired polymerization of the acrylate can also be minimized through the choice of acrylate.

The tertiary alcohol of formula (I) prepared by the method according to the present invention can then be converted to the end product 2-cyclododecyl-1-propanol in further reaction steps which are known per se, as described, for example, in DE 37 03 585 A1.

The method according to the present invention is described in more detail under reference to the following example, which is provided for illustration only and which is not intended to limit the scope of protection of the present invention.

### EXAMPLE

A 2-litre three-necked flask with a magnetic stirrer and a dropping funnel was inerted with N₂ by means of three vacuum-purge cycles. 1070 ml toluene was added in the N₂ countercurrent flow, followed by 2.04 g of hydroquinone monomethyl ether, 4.93 g of Cu(OAc)₂×H₂O, and 300 g of (powdered) cyclododecanone. 16.19 g of triphenylphosphine was then added.

225.3 g of PMHS was then added slowly at room temperature using a dropping funnel. The mixture was then stirred at room temperature for 15 minutes.

The batch was then heated to 30° C., and 173 g ethyl acrylate was slowly added dropwise using a dropping funnel. The reaction started after the addition of approximately 5 g of ethyl acrylate, as was evident from a rise in the internal temperature. During the further dropwise addition of the ethyl acrylate over a period of 3.5 h, the internal temperature of the batch was held between 25° C. and 35° C. (the temperature being controlled with a water bath to the extent necessary). Once all the ethyl acrylate was added, the batch was stirred for 18 h at room temperature.

500 ml of concentrated HCl (32%) was added dropwise over a period of 2 h to the polymethylsiloxane compound that was formed as an intermediate, without work-up, in order to obtain the corresponding tertiary alcohol. The internal temperature initially rose from 22° ° C. to 25° C. Once all the HCl was added, the reaction batch was stirred for 18 h at room temperature. The batch was transferred to a separating funnel and the phases separated.

A small sample of the tertiary alcohol was taken for characterization and analyzed by GC. This showed that the tertiary alcohol formed was identical to the tertiary alcohol obtained from a corresponding Reformatsky reaction of cyclododecanone and ethyl 2-bromopropionate in the presence of zinc, in accordance with DE 3703585 A1 (Example 1, but without elimination of water from the tertiary alcohol).

Characterization data: In addition to the desired tertiary alcohol, still unreacted cyclododecanone, cyclododecanol and triphenylphosphine along with further, unknown substances could be detected in the GC-FID analysis.

The features of the present invention as disclosed in above can be essential both individually and in any combination with one another for the realization of the present invention in its various embodiments. Reference should also be had to the appended claims.

What is claimed is:

1-14. (canceled)

15. A method for preparing a tertiary alcohol having a formula (I) 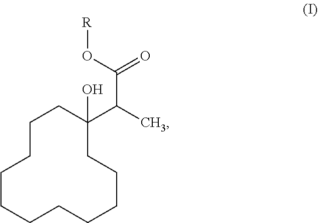
the method comprising:
reacting cyclododecanone with an acrylate having a formula RO—CO—CH═CH₂ in a reductive aldol reaction under a metal catalysis,
wherein the R in the formula (I) is,
a straight-chain C₁-C₈-alkyl,
a branched C₁-C₈-alkyl,
a straight-chain alkenyl group,
a branched alkenyl group,
an unsubstituted phenyl group, or
a substituted phenyl group.

16. The method as recited in claim 15, wherein,
the metal catalysis is a copper catalysis, and
the reacting is further performed in a presence of a silane.

17. The method as recited in claim 16, wherein the silane is polymethylhydrosiloxane (PMHS).

18. The method as recited in claim 16, wherein Cu(OAc)₂×H₂O is used as a catalyst in the copper catalysis.

19. The method as recited in claim 18, wherein a phosphine compound is used as a ligand of the catalyst.

20. The method as recited in claim 19, wherein triphenylphosphine is used as the phosphine compound.

21. The method as recited in claim 15, wherein a stabilizer is used for the acrylate.

22. The method as recited in claim 21, wherein the stabilizer is hydroquinone monomethyl ether.

23. The method as recited in claim 15, wherein the reacting is performed in a solvent system.

24. The method as recited in claim 23, wherein the solvent system includes toluene or xylene.

25. The method as recited in claim 15, wherein the reacting is performed at a temperature of from −30° C. to 80° C.

26. The method as recited in claim 15, wherein the reacting is performed at a temperature of from 20° C. to 30° C.

27. The method as recited in claim 15, wherein R in the formula (I) is,
a straight-chain C₁-C₈-alkyl,
a branched C₁-C₈-alkyl,
a straight-chain alkenyl group, or
a branched alkenyl group.

28. The method as recited in claim 27, wherein R is an ethyl group, an n-butyl group or a 2-ethylhexyl group.

29. A method of preparing 2-cyclododecyl-1-propanol, the method comprising:
providing the tertiary alcohol having the formula (I) as recited in claim 15; and
converting the tertiary alcohol having the formula (I) to 2-cyclododecyl-1-propanol.
